(19) United States
(12) Patent Application Publication
(10) Pub. No.: US 2024/0424008 A1
(43) Pub. Date: Dec. 26, 2024

(54) Compound Tulathromycin Nanoemulsion, and Preparation Method and Use Thereof

(71) Applicants: Hubei Wudang Animal Pharmaceutical Co., Ltd, Shiyan City (CN); Wuhan Academy of Agricultural Sciences, Wuhan City (CN)

(72) Inventors: Bin HE, Wuhan City (CN); Li CHEN, Wuhan City (CN); Guoming CHEN, Wuhan City (CN); Lijun WU, Wuhan City (CN); Tianzhong TANG, Wuhan City (CN)

(73) Assignees: Hubei Wudang Animal Pharmaceutical Co., Ltd, Shiyan City (CN); Wuhan Academy of Agricultural Sciences, Wuhan City (CN)

(21) Appl. No.: 18/540,842

(22) Filed: Dec. 14, 2023

(30) Foreign Application Priority Data
Jun. 20, 2023 (CN) 202310728657.9

(51) Int. Cl. A61K 31/7052; A61K 9/107; A61K 31/365; A61K 47/10; A61K 47/14; A61K 47/26

(52) U.S. Cl. CPC A61K 31/7052; A61K 9/1075; A61K 31/365; A61K 47/10; A61K 47/14; A61K 47/26

(57) ABSTRACT

Provided are a compound tulathromycin nanoemulsion, and a preparation method and use thereof, which belong to the technical field of nanoemulsions. The compound tulathromycin nanoemulsion includes, in percentages by mass: 0.01% to 10.0% of tulathromycin, 0.01% to 1.0% of kalii dehydrographolidi succinas, 1.0% to 5.0% of ethyl acetate, 0.5% to 3.5% of ethyl butyrate, 0.5% to 2.5% of tributyl citrate, 1.0% to 5.0% of ethanol, 10.0% to 15.0% of polyethylene glycol sorbitan monostearate, 6.0% to 12.0% of octylphenol polyoxyethylene(10) ether, and water as balance.

### CROSS REFERENCE TO RELATED APPLICATION

This patent application claims the benefit and priority of Chinese Patent Application No. 2023107286579 filed with the China National Intellectual Property Administration on Jun. 20, 2023, and entitled “Compound tulathromycin nanoemulsion, and a preparation method and use thereof”, the disclosure of which is incorporated by reference herein in its entirety as part of the present application.

### TECHNICAL FIELD

The present disclosure relates to the technical field of nanoemulsions, in particular to a compound tulathromycin nanoemulsion, and a preparation method and use thereof.

### BACKGROUND

Tulathromycin is a new type of macrolide drug, and is mainly used clinically for the treatment of respiratory diseases in livestock and poultry. However, tulathromycin has strong fat solubility and extremely low solubility in water. Therefore, when the tulathromycin is prepared into preparations such as injections and oral solutions, a large amount of organic solvents with higher solubility are required to dissolve the tulathromycin. Compared with aqueous solvents, organic solvents are either oily lipid substances or other organic pharmaceutical auxiliary materials with higher solubility, and therefore significantly increase the cost of drugs than those with water as the auxiliary materials. For example, the commercially available 10% tulathromycin injection contains only 10 wt % of the active ingredients, and the other 90 wt % is a relatively high-cost organic solvent (having a moisture content within 1.0 wt %), leading to that there is great hindrance in the promotion of this drug when being used in farmed animals. In addition, the extensive use of organic solvents may also cause great harm to the animal body, such as hemolysis, local suppuration, and inflammation. The animals always show local redness and swelling after being administrated some drug products, and intramuscular injection of the leg may also cause lameness in animals. After some animals are slaughtered, pustules may appear at the injection site, adversely affecting the quality of meat.

### SUMMARY

An object of the present disclosure is to provide a compound tulathromycin nanoemulsion, and a preparation method and use thereof. In the present disclosure, the compound tulathromycin nanoemulsion has high stability, desirable safety, and low cost.

To achieve the above object, the present disclosure provides the following technical solutions.

The present disclosure provides a compound tulathromycin nanoemulsion, including, in percentages by mass,
0.01% to 10.0% of tulathromycin, 0.01% to 1.0% of kalii dehydrographolidi succinas, 1.0% to 5.0% of ethyl acetate, 0.5% to 3.5% of ethyl butyrate, 0.5% to 2.5% of tributyl citrate, 1.0% to 5.0% of ethanol, 10.0% to 15.0% of polyethylene glycol sorbitan monostearate (tween-60), 6.0% to 12.0% of octylphenol polyoxyethylene (10) ether (op-10), and water as balance.

In some embodiments, the compound tulathromycin nanoemulsion includes, in percentages by mass,
1.0% to 9.0% of the tulathromycin, 0.1% to 0.9% of the kalii dehydrographolidi succinas, 2.0% to 4.0% of the ethyl acetate, 1.0% to 3.0% of the ethyl butyrate, 1.0% to 2.0% of the tributyl citrate, 2.0% to 4.0% of the ethanol, 11.0% to 14.0% of the tween-60, 7.0% to 11.0% of the op-10, and water as balance.

In some embodiments, the compound tulathromycin nanoemulsion consists of, in percentages by mass,
5.0% of the tulathromycin, 0.5% of the kalii dehydrographolidi succinas, 3.0% of the ethyl acetate, 2.0% of the ethyl butyrate, 1.5% of the tributyl citrate, 3.0% of the ethanol, 12.5% of the tween-60, 9.0% of the op-10, and 63.5% of water.

In some embodiments, the compound tulathromycin nanoemulsion is an oil-in-water nanoemulsion; and drug particles in the compound tulathromycin nanoemulsion have a particle size of 4 nm to 20 nm.

The present disclosure further provides a method for preparing the compound tulathromycin nanoemulsion, including the following steps:
subjecting the ethyl acetate, the ethyl butyrate, the tributyl citrate, the tulathromycin, and the kalii dehydrographolidi succinas to first mixing to obtain a first mixed feed liquid;
subjecting the tween-60, the op-10, and the ethanol to second mixing to obtain a second mixed feed liquid;
subjecting the first mixed feed liquid, the second mixed feed liquid, and the water to third mixing to obtain a third mixed feed liquid; and
subjecting the third mixed feed liquid to emulsification to obtain the compound tulathromycin nanoemulsion.

In some embodiments, the first mixing includes:
mixing the ethyl acetate, the ethyl butyrate, and the tributyl citrate to obtain a oil phase, and adding the tulathromycin and the kalii dehydrographolidi succinas into the oil phase.

In some embodiments, the second mixing specifically includes:
mixing the tween-60 and the op-10, and adding the ethanol thereto.

In some embodiments, the third mixing includes:
adding the second mixed feed liquid into the first mixed feed liquid to obtain a mixture, and adding the water into the mixture.

In some embodiments, the emulsification is conducted at a rotation speed of 2,000 r/min to 5,000 r/min for 3 min to 5 min.

The present disclosure further provides use of the compound tulathromycin nanoemulsion as described in above technical solutions or the compound tulathromycin nanoemulsion prepared by the method as described in above technical solutions in preparation of a drug for treating a respiratory disease.

The present disclosure provides a compound tulathromycin nanoemulsion, including in percentages by mass: 0.01% to 10.0% of tulathromycin, 0.01% to 1.0% of kalii dehydrographolidi succinas, 1.0% to 5.0% of ethyl acetate, 0.5% to 3.5% of ethyl butyrate, 0.5% to 2.5% of tributyl citrate, 1.0% to 5.0% of ethanol, 10.0% to 15.0% of tween-60, 6.0% to 12.0% of op-10, and water as balance. In the present disclosure, kalii dehydrographolidi succinas is introduced into the compound tulathromycin nanoemulsion. The kalii dehydrographolidi succinas is a traditional Chinese medicine ingredient extracted from the traditional Chinese medicinal material Andrographis Paniculata, and has Immune Stimulation, Anti-Infection, and Anti-Inflammatory effects. This ingredient is mainly used for adjuvant treatment of viral pneumonia and viral upper respiratory infection, and has the effects of significantly relieving dyspnea, promoting gas oxygen exchange, and antipyretic. This ingredient can be used for the treatment of upper respiratory infection, especially acute bronchitis and other diseases. The kalii dehydrographolidi succinas and the tulathromycin are combined to prepare the compound tulathromycin nanoemulsion. These two ingredients complement with each other and do not affect drug stability after compatibility of drugs. Moreover, the combination of Chinese and Western medicine shows a better effect, such that the compound tulathromycin nanoemulsion has a wider antipathogenic spectrum (including viruses, bacteria, and mycoplasma), a significantly enhanced efficacy, and a significantly-shortened treatment duration. The nanoemulsion also has a more thorough treatment effect on respiratory diseases and has more promotion advantages than single tulathromycin injection. Moreover, since the compound tulathromycin nanoemulsion is a water-containing preparation with a high proportion of water (not less than 46 wt %), the cost of auxiliary materials is significantly reduced and the safety is also significantly improved. In clinical applications, the compound tulathromycin nanoemulsion has desirable water solubility, and could be infinitely diluted with water without demulsification, layering, settling, or precipitation; moreover, the nanoemulsion has great stability, which reduces the hindrance of tulathromycin-based drugs in veterinary clinical promotion.

### BRIEF DESCRIPTION OF THE DRAWINGS

FIG. 1 shows measured particle size distribution of the compound tulathromycin nanoemulsion in Example 1.

FIG. 2 shows an electron micrograph of the compound tulathromycin nanoemulsion in Example 1.

### DETAILED DESCRIPTION OF THE EMBODIMENTS

The present disclosure provides a compound tulathromycin nanoemulsion, including in percentages by mass:
0.01% to 10.0% of tulathromycin, 0.01% to 1.0% of kalii dehydrographolidi succinas, 1.0% to 5.0% of ethyl acetate, 0.5% to 3.5% of ethyl butyrate, 0.5% to 2.5% of tributyl citrate, 1.0% to 5.0% of ethanol, 10.0% to 15.0% of tween-60, 6.0% to 12.0% of op-10, and water as balance.

In the present disclosure, unless otherwise specified, all raw materials required for preparation are commercially available products well known to persons skilled in the art.

In the present disclosure, the compound tulathromycin nanoemulsion includes 0.01% to 10.0% by mass, preferably 1.0% to 9.0% by mass, and more preferably 5.0% by mass of the tulathromycin.

In the present disclosure, the compound tulathromycin nanoemulsion includes 0.01% to 1.0% by mass, preferably 0.1% to 0.9% by mass, and more preferably 0.5% by mass of the kalii dehydrographolidi succinas.

The kalii dehydrographolidi succinas and the tulathromycin are combined to prepare the compound tulathromycin nanoemulsion. These two ingredients complement with each other and do not affect drug stability after compatibility of drugs. Moreover, the combination of Chinese and Western medicines shows a better effect, such that the compound tulathromycin nanoemulsion has a wider antipathogenic spectrum (including viruses, bacteria, and mycoplasma), a significantly enhanced efficacy, and a significantly-shortened treatment duration. The nanoemulsion also has a more thorough treatment effect on respiratory diseases and has more promotion advantages than single tulathromycin injection.

In the present disclosure, the compound tulathromycin nanoemulsion includes 1.0% to 5.0% by mass, preferably 2.0% to 4.0% by mass, and more preferably 3.0% by mass of the ethyl acetate.

In the present disclosure, the compound tulathromycin nanoemulsion includes 0.5% to 3.5% by mass, preferably 1.0% to 3.0% by mass, and more preferably 2.0% by mass of the ethyl butyrate.

In the present disclosure, the compound tulathromycin nanoemulsion includes 0.5% to 2.5% by mass, preferably 1.0% to 2.0% by mass, and more preferably 1.5% by mass of the tributyl citrate.

In the present disclosure, the ethyl acetate, ethyl butyrate, and tributyl citrate are compounded and used as an oil phase, and the oil phase has advantages of safety during use, high fluidity, and desirable syringeability; also, tulathromycin has a high solubility in the oil phase. In contrast, though some oily lipids (such as soybean oil, sesame oil, and camellia oil) could achieve desirable solubility of tulathromycin, they exhibit poor syringeability and inconvenient clinical injection. If one of ethyl acetate, ethyl butyrate, and tributyl citrate is used alone, the solubility of tulathromycin therein is low, and the prepared compound tulathromycin nanoemulsion thereby has a low drug-loading capacity. In the present disclosure, compounding the ethyl acetate, ethyl butyrate, and tributyl citrate could significantly increase the drug-loading capacity (specifically referring to the percentage content of tulathromycin and kalii dehydrographolidi succinas), and the prepared compound tulathromycin nanoemulsion is more stable.

In the present disclosure, the compound tulathromycin nanoemulsion includes 1.0% to 5.0% by mass, preferably 2.0% to 4.0% by mass, and more preferably 3.0% by mass of the ethanol. The ethanol is preferably absolute ethanol. On one hand, the ethanol could be used as a co-surfactant to help the system emulsify, improve emulsification efficiency, and reduce production time; on the other hand, tulathromycin also has certain solubility in the ethanol, which is beneficial to maintaining the stability of the system and preventing the drug from precipitating after being left for a long time.

In the present disclosure, the compound tulathromycin nanoemulsion includes 10.0% to 15.0% by mass, preferably 11.0% to 14.0% by mass, and more preferably 12.5% by mass of the tween-60.

In the present disclosure, the compound tulathromycin nanoemulsion includes 6.0% to 12.0% by mass, preferably 7.0% to 11.0% by mass, and more preferably 9.0% by mass of the op-10. The op-10 refers to octylphenol polyoxyethylene(10) ether.

In the present disclosure, the tween-60 and op-10 are used in combination as the emulsifier, which could have a better emulsification effect on the oil phase formed by the ethyl acetate, ethyl butyrate, and tributyl citrate. Specifically, when a hydrophilic-lipophilic balance (HLB) value required for emulsification of the oil phase is consistent with the HLB value of the emulsifier itself, there may be a better emulsification effect. The tween-60 and op-10 are used as the emulsifier, which show great process operability and easiness in production and conversion, thereby avoiding that the fluidity is too low during emulsification to uniformly emulsify, since some emulsifiers (such as span 80, tween-80, sucrose fatty acid ester, and polyoxyethylene ether-hydrogenated castor oil 40) are too viscous. If tween-60 or op-10 is used alone, demulsification is prone to occur, resulting in that the compound tulathromycin nanoemulsion is prone to layer, become turbid, and even that drug precipitates out from the nanoemulsion, thus showing poor stability.

In the present disclosure, the compound tulathromycin nanoemulsion includes water as balance. In some embodiments, the water is distilled water.

In some embodiments of the present disclosure, drug particles in the compound tulathromycin nanoemulsion are in the shape of a regular sphere. In some embodiments, the drug particles have a particle size of 4 nm to 20 nm, preferably 5 nm to 18 nm, and even more preferably 6 nm to 17 nm.

The present disclosure further provides a method for preparing the compound tulathromycin nanoemulsion, including the following steps:
subjecting the ethyl acetate, the ethyl butyrate, the tributyl citrate, the tulathromycin, and the kalii dehydrographolidi succinas to first mixing to obtain a first mixed feed liquid;
subjecting the tween-60, the op-10, and the ethanol to second mixing to obtain a second mixed feed liquid;
subjecting the first mixed feed liquid, the second mixed feed liquid, and water to third mixing to obtain a third mixed feed liquid; and
subjecting the third mixed feed liquid to emulsification to obtain the compound tulathromycin nanoemulsion.

In the present disclosure, the ethyl acetate, the ethyl butyrate, the tributyl citrate, the tulathromycin, and the kalii dehydrographolidi succinas are subjected to first mixing to obtain a first mixed feed liquid. In some embodiments, the first mixing includes: mixing the ethyl acetate, the ethyl butyrate, and the tributyl citrate to obtain an oil phase, and adding the tulathromycin and the kalii dehydrographolidi succinas into the oil phase.

In the present disclosure, the tween-60, the op-10, and the ethanol are subjected to second mixing to obtain a second mixed feed liquid. In some embodiments, the second mixing includes: mixing the tween-60 and the op-10, and adding the ethanol thereto.

In the present disclosure, after obtaining the first mixed feed liquid and the second mixed feed liquid, the first mixed feed liquid, the second mixed feed liquid, and the water are subjected to third mixing to obtain a third mixed feed liquid. In some embodiments, the third mixing includes: adding the second mixed feed liquid into the first mixed feed liquid to obtain a mixture, and adding the water into the mixture.

In some embodiments of the present disclosure, the first mixing, the second mixing, and the third mixing each are conducted under stirring. In the present disclosure, there is no special limitation on the stirring, as long as the ingredients are mixed uniformly.

In the present disclosure, after obtaining the third mixed feed liquid, the third mixed feed liquid is subjected to emulsification to obtain the compound tulathromycin nanoemulsion. In some embodiments, the emulsification is conducted at a rotation speed of 2,000 r/min to 5,000 r/min, preferably 2,500 r/min to 4,500 r/min, and even more preferably 3500 r/min. In some embodiments, the emulsification is conducted for 3 min to 5 min, preferably 3.5 min to 4.5 min, and even more preferably 4 min. In some embodiments, the emulsification is conducted in a homogenizer. The compound tulathromycin nanoemulsion is a uniform and transparent yellow liquid with desirable fluidity.

In the present disclosure, tulathromycin and kalii dehydrographolidi succinas are combined and prepared into an oil-in-water nanoemulsion, and drug particles in the nanoemulsion have a particle size of 4 nm to 20 nm, which greatly reduces the dosage of organic solvent used, and significantly improves the bioavailability and therapeutic effect of drugs. Specifically, the kalii dehydrographolidi succinas is a traditional Chinese medicine extract that has immune stimulation, anti-infection, and anti-inflammation effects, and is biased towards symptomatic treatment; and the tulathromycin belongs to western medicine, has extremely strong antibacterial effect, and is biased towards etiological treatment. The combination of kalii dehydrographolidi succinas and tulathromycin belongs to the combination of Chinese and Western medicines, and also “symptomatic treatment”+“etiological treatment”; and the above two have no incompatibility, and show a more ideal therapeutic effect than that of a single ingredient. Nanoemulsions have advantages of targeting and small size effects. The targeting effect is manifested by bringing the drug to nidus(es) through inflammatory chemotaxis and the ability of leukocytes to phagocytose nanomaterials; the small size effect is manifested in that after the drug particles become smaller, they are easily absorbed through the intestinal mucosa when administrated orally, and when administrated by injection, the nanoemulsions are more likely to penetrate the envelope, enter the pathogenic cytoplasm when encountering pathogens, and cause the death of pathogenic bacteria by preventing protein synthesis. Since the compound tulathromycin nanoemulsion is a water-containing preparation with a high proportion of water (not less than 46 wt %), the cost is significantly reduced while the safety is also significantly improved. The compound tulathromycin nanoemulsion has desirable solubility in water, and could be infinitely diluted with water without demulsification, layering, settling, or precipitation; moreover, the nanoemulsion has good stability, strong operability in preparation process, and easiness in production and transformation, which significantly reduce the hindrance in the veterinary clinical promotion of tulathromycin-based drugs.

The present disclosure further provides use of the compound tulathromycin nanoemulsion as described in above technical solutions or the compound tulathromycin nanoemulsion prepared by the method as described in above technical solutions in preparation of a drug for treating a respiratory disease. In the present disclosure, the compound tulathromycin nanoemulsion has desirable stability, and could be administrated in drinking water (that is to say, administrated after being diluted with water) in addition to injection, thus providing flexible administration manners. In some embodiments, the drug for treating the respiratory disease is a drug for treating a respiratory disease of livestock and poultry.

The technical solutions of the present disclosure will be clearly and completely described below in conjunction with the examples of the present disclosure. Apparently, the described examples are merely a part rather than all of the examples of the present disclosure. All other embodiments obtained by those skilled in the art based on the embodiments of the present disclosure without creative efforts shall fall within the scope of the present disclosure.

### Example 1

3.0 g of ethyl acetate, 2.0 g of ethyl butyrate, and 1.5 g of tributyl citrate were mixed to prepare an oil phase. 5.0 g of tulathromycin and 0.5 g of kalii dehydrographolidi succinas were added into the oil phase, and a resulting mixture was stirred to dissolve the tulathromycin and kalii dehydrographolidi succinas in the oil phase, obtaining a first mixed feed liquid.

12.5 g of tween-60 and 9.0 g of op-10 were mixed evenly by stirring, and 3.0 g of absolute ethanol was added thereto. They were then mixed evenly by stirring, obtaining a second mixed feed liquid.

The second mixed feed liquid was added into the first mixed feed liquid and they were mixed evenly by stirring, obtaining a mixture. 63.5 g of distilled water was then added into the mixture and a resulting mixture was mixed evenly by stirring, obtaining a third mixed feed liquid.

The third mixed feed liquid was poured into a homogenizer, and subjected to emulsification under high-speed shearing at a rotation speed of 3,500 r/min for 4 min, such that a resulting system became a uniform and transparent yellow liquid with desirable fluidity, obtaining the compound tulathromycin nanoemulsion.

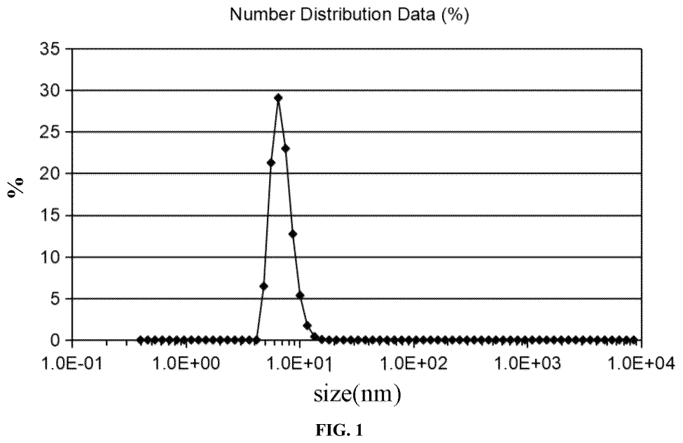

A particle size of drug particles in the compound tulathromycin nanoemulsion in Example 1 was determined with a laser particle size analyzer. The results are shown in FIG. 1. As shown in FIG. 1, drug particles in the compound tulathromycin nanoemulsion in Example 1 have a particle size of 4 nm to 20 nm.

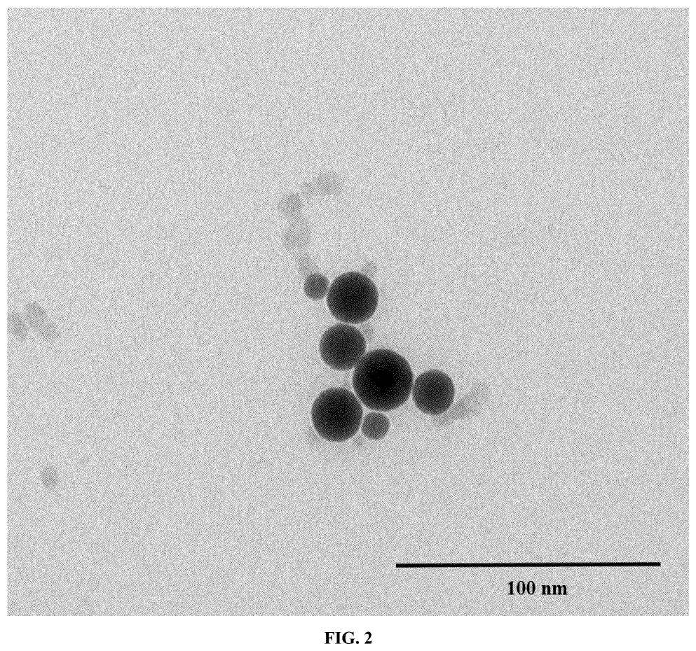

The compound tulathromycin nanoemulsion in Example 1 was observed with an electron microscope. The results are shown in FIG. 2. As shown in FIG. 2, the drug particles in the compound tulathromycin nanoemulsion in Example 1 are in the form of regular spherical bodies (the spherical bodies are an oil-in-water emulsion).

### Example 2

1.0 g of ethyl acetate, 0.5 g of ethyl butyrate, and 0.5 g of tributyl citrate were mixed to prepare an oil phase. 0.01 g of tulathromycin and 0.01 g of kalii dehydrographolidi succinas were added into the oil phase. A resulting mixture was stirred to dissolve the tulathromycin and kalii dehydrographolidi succinas in the oil phase, obtaining a first mixed feed liquid.

10.0 g of tween-60 and 6.0 g of op-10 were mixed evenly by stirring, and 1.0 g of absolute ethanol was added thereto. They were then mixed evenly by stirring, obtaining a second mixed feed liquid.

The second mixed feed liquid was added into the first mixed feed liquid and they were mixed evenly by stirring, obtaining a mixture. 80.98 g of distilled water was then added into the mixture and a resulting mixture was mixed evenly by stirring, obtaining a third mixed feed liquid.

The third mixed feed liquid was poured into a homogenizer, and subjected to emulsification under high-speed shearing at a rotation speed of 2,500 r/min for 3.5 min, such that a resulting system became a uniform and transparent yellow liquid with desirable fluidity, obtaining the compound tulathromycin nanoemulsion.

In this example, drug particles in the compound tulathromycin nanoemulsion were in the shape of a regular sphere, and the drug particles had a particle size of 6 nm to 17 nm.

### Example 3

5.0 of ethyl acetate, 3.5 g of ethyl butyrate, and 2.5 g of tributyl citrate were mixed to prepare an oil phase. 10.0 g of tulathromycin and 1.0 g of kalii dehydrographolidi succinas were added into the oil phase, and a resulting mixture was stirred to dissolve the tulathromycin and kalii dehydrographolidi succinas in the oil phase, obtaining a first mixed feed liquid.

15.0 g of tween-60 and 12.0 g of op-10 were mixed evenly by stirring, and 5.0 g of absolute ethanol was added thereto. They were mixed evenly by stirring, obtaining a second mixed feed liquid.

The second mixed feed liquid was added into the first mixed feed liquid and they were mixed evenly by stirring, obtaining a mixture. 46.0 g of distilled water was then added into the mixture and a resulting mixture was mixed evenly by stirring, obtaining a third mixed feed liquid.

The third mixed feed liquid was poured into a homogenizer, and subjected to emulsification under high-speed shearing at a rotation speed of 4,500 r/min for 4.5 min, such that a resulting system became a uniform and transparent yellow liquid with desirable fluidity, obtaining the compound tulathromycin nanoemulsion.

In this example, drug particles in the compound tulathromycin nanoemulsion were in the shape of a regular sphere, and the drug particles had a particle size of 5 nm to 18 nm.

### Performance Testing

A common competing product on the market, tulathromycin injection “Draxxin™”, was mixed with water at a volume ratio (tulathromycin injection “Draxxin™”/water) of 1:1, 1:2, 1:4, 1:8, 1:16, 1:32, 1:64, 1:128, 1:256, 1:512, and 1:1024, and resulting mixtures were then placed at room temperature (25° C.). Drugs precipitated from each sample within a few minutes, and a higher dilution factor led to a greater amount of drug precipitated. When the tulathromycin injection and water were mixed at a volume ratio of 1:64, all the drugs were precipitated; and the amount of drug precipitated at a volume ratio of 1:128, 1:256, 1:512, and 1:1024 was the same as that of 1:64, that is to say, all drugs were completely precipitated.

The compound tulathromycin nanoemulsions prepared in Examples 1 to 3 of the present disclosure were separately mixed with water at a volume ratio (compound tulathromycin nanoemulsion/water) of 1:1, 1:2, 1:4, 1:8, 1:16, 1:32, 1:64, 1:128, 1:256, 1:512, and 1:1024, and resulting mixtures were then placed at room temperature for continuous 24 h. There were no phenomena such as demulsification, layering, and drug precipitation in each sample.

From the above results, it can be known that the compound tulathromycin nanoemulsion in the present disclosure has desirable stability. Based on this, the compound tulathromycin nanoemulsion could not only be administrated through injection, but also could be administrated in drinking water, showing flexible administration manners. However, when diluting the common commercially available competing product tulathromycin injections with water, the drug precipitates, making it unsuitable to be administrated in drinking water.

The above descriptions are merely preferred embodiments of the present disclosure. It should be noted that a person of ordinary skill in the art may further make several improvements and modifications without departing from the principle of the present disclosure, but such improvements and modifications should be deemed as falling within the scope of the present disclosure.

What is claimed is:

1. A compound tulathromycin nanoemulsion, comprising, in percentages by mass,
0.01% to 10.0% of tulathromycin, 0.01% to 1.0% of kalii dehydrographolidi succinas, 1.0% to 5.0% of ethyl acetate, 0.5% to 3.5% of ethyl butyrate, 0.5% to 2.5% of tributyl citrate, 1.0% to 5.0% of ethanol, 10.0% to 15.0% of polyethylene glycol sorbitan monostearate, 6.0% to 12.0% of octylphenol polyoxyethylene(10) ether, and water as balance.

2. The compound tulathromycin nanoemulsion as claimed in claim 1, wherein the compound tulathromycin nanoemulsion comprises, in percentages by mass,
1.0% to 9.0% of the tulathromycin, 0.1% to 0.9% of the kalii dehydrographolidi succinas, 2.0% to 4.0% of the ethyl acetate, 1.0% to 3.0% of the ethyl butyrate, 1.0% to 2.0% of the tributyl citrate, 2.0% to 4.0% of the ethanol, 11.0% to 14.0% of the polyethylene glycol sorbitan monostearate, 7.0% to 11.0% of the octylphenol polyoxyethylene(10) ether, and water as balance.

3. The compound tulathromycin nanoemulsion as claimed in claim 2, wherein the compound tulathromycin nanoemulsion consists of, in percentages by mass,
5.0% of the tulathromycin, 0.5% of the kalii dehydrographolidi succinas, 3.0% of the ethyl acetate, 2.0% of the ethyl butyrate, 1.5% of the tributyl citrate, 3.0% of the ethanol, 12.5% of the polyethylene glycol sorbitan monostearate, 9.0% of the octylphenol polyoxyethylene(10) ether, and 63.5% of the water.

4. The compound tulathromycin nanoemulsion as claimed in claim 1, wherein the compound tulathromycin nanoemulsion is an oil-in-water nanoemulsion; and drug particles in the compound tulathromycin nanoemulsion have a particle size of 4 nm to 20 nm.

5. The compound tulathromycin nanoemulsion as claimed in claim 2, wherein the compound tulathromycin nanoemulsion is an oil-in-water nanoemulsion; and drug particles in the compound tulathromycin nanoemulsion have a particle size of 4 nm to 20 nm.

6. The compound tulathromycin nanoemulsion as claimed in claim 3, wherein the compound tulathromycin nanoemulsion is an oil-in-water nanoemulsion; and drug particles in the compound tulathromycin nanoemulsion have a particle size of 4 nm to 20 nm.

7. A method for preparing the compound tulathromycin nanoemulsion as claimed in claim 1, comprising the steps of subjecting the ethyl acetate, the ethyl butyrate, the tributyl citrate, the tulathromycin, and the kalii dehydrographolidi succinas to first mixing to obtain a first mixed feed liquid;
subjecting the polyethylene glycol sorbitan monostearate, the octylphenol polyoxyethylene(10) ether, and the ethanol to second mixing to obtain a second mixed feed liquid;
subjecting the first mixed feed liquid, the second mixed feed liquid, and the water to third mixing to obtain a third mixed feed liquid; and
subjecting the third mixed feed liquid to emulsification to obtain the compound tulathromycin nanoemulsion.

8. The method as claimed in claim 7, wherein the first mixing comprises:
mixing the ethyl acetate, the ethyl butyrate, and the tributyl citrate to obtain an oil phase, and adding the tulathromycin and the kalii dehydrographolidi succinas into the oil phase.

9. The method as claimed in claim 7, wherein the second mixing comprises:
mixing the polyethylene glycol sorbitan monostearate and the octylphenol polyoxyethylene(10) ether, and adding the ethanol thereto.

10. The method as claimed in claim 7, wherein the third mixing comprises:
adding the second mixed feed liquid into the first mixed feed liquid to obtain a mixture, and adding the water into the mixture.

11. The method as claimed in claim 7, wherein the emulsification is conducted at a rotation speed of 2,000 r/min to 5,000 r/min for 3 min to 5 min.

12. The method as claimed in claim 8, wherein the emulsification is conducted at a rotation speed of 2,000 r/min to 5,000 r/min for 3 min to 5 min.

13. The method as claimed in claim 9, wherein the emulsification is conducted at a rotation speed of 2,000 r/min to 5,000 r/min for 3 min to 5 min.

14. The method as claimed in claim 10, wherein the emulsification is conducted at a rotation speed of 2,000 r/min to 5,000 r/min for 3 min to 5 min.

15. A method for treating a respiratory disease, comprising the step of
administrating the compound tulathromycin nanoemulsion as claimed in claim 1 through digestion, or
administrating the compound tulathromycin nanoemulsion as claimed in claim 1 after being diluted with water.
